(19) United States
(12) Patent Application Publication
(10) Pub. No.: US 2023/0235259 A1
(43) Pub. Date: Jul. 27, 2023

(54) Flow Path Cassette, Cleaning Kit, And Cell Cleaning System

(71) Applicants: Terumo Kabushiki Kaisha, Tokyo (JP); Terumo BCT, Inc., Lakewood, CO (US)

(72) Inventors: Masatsugu IGARASHI, Fujinomiya-shi (JP); Briden Ray STANTON, Highlands Ranch, CO (US); Jon A. DODD, Littleton, CO (US)

(21) Appl. No.: 18/090,149

(22) Filed: Dec. 28, 2022

Related U.S. Application Data
(63) Continuation of application No. PCT/JP2021/024304, filed on Jun. 28, 2021.

(30) Foreign Application Priority Data
Jul. 2, 2020 (JP) 2020-114572

(51) Int. Cl. C12M 1/12; A61M 1/36; C12M 1/00; C12M 3/00; C12M 1/26

(52) U.S. Cl. CPC C12M 23/06; A61M 1/3622; C12M 23/26; C12M 23/42; C12M 23/44; C12M 33/04; C12M 47/02; C12M 23/40

(57) ABSTRACT

A flow path cassette includes superimposed first and second flexible sheets, where a plurality of flow paths are disposed therebetween and detection channel portions are disposed at one or more points along one or more of the plurality the flow paths. Each of the detection channel portions includes a first bulging portion and an opposing second bulging portion. A plate member is aligned with the second bulging portion, and a deformation preventative member is aligned with the first bulging portion. The plate member may move with the second bulging portion, while the deformation preventative member prevents deformation of the first bulging portion.

### CROSS-REFERENCE TO RELATED APPLICATIONS

The present application is a continuation application of International Patent Application No. PCT/JP2021/024304 filed on Jun. 28, 2021, which designated the U.S. and claims the benefit of priority from Japanese Patent Application No. JP2020-114572 filed Jul. 2, 2020. The entire disclosures of the above-identified applications are incorporated herein by reference.

### FIELD

The present invention relates to a flow path cassette, a cleaning kit, and a cell cleaning system.

### BACKGROUND

Regenerative medicine involves treatments that often include administering to patients cultured therapeutic cells, including, for example, embryonic steam (ES) cells, induced pluripotent stem (iPS) cells, and/or mesenchymal stem cells.

Culturing therapeutic cells for regenerative medicine often include using a cell culture system that, as described in Japanese Laid-Open Patent Publication No. 2017-143775, may include a bioreactor having a hollow fiber. The cell culture system may include a plurality of bags, including, for example, one or more call bags where cells to be inoculated are accommodated, one or more medium bags where a medium (i.e., culture solution) is accommodated, one or more waste liquid bags where a waste liquid is accommodated, and/or one or more peeling solution bags where peeling cells from a flow path during a cell harvesting process are accommodated. A complex assembly of connectors (e.g., tubes) is often needed to connect the plurality of bags.

In various aspects, cultured cells may be subjected to one or more cleaning processes prior to use for regenerative medicine. The cleaning processes may remove excess medium, as well as other foreign substances, to increase cell concentration. The cultured cells may be cleaned using a cleaning system and process as described, for example, in Japanese Laid-Open Patent Publication No. 2015-188315. Like the cell culture system, the cleaning system may include a plurality of bags, including, for example, one or more culture bags, one or more cleaning solution bags, and/or one or more product bags, and a complex assembly of connectors (e.g., tubes) is often needed to connect the plurality of bags.

In each instance, coupling the plurality of connectors and constructing the complex assemblies is often complicated and time consuming. Accordingly, it may be desirable to develop systems and products that address these challenges.

### SUMMARY

The present disclosure relates to disposable and replaceable cassettes including flow paths disposed between a pair of flexible sheets and means for measuring internal pressures of the flow paths. The present disclosure also relates to cleaning kits that include such disposable flow path cassettes and to cell cleaning systems that include such cleaning kits.

Various aspects of the present disclosure relate to a flow path cassette (also referred to as a cassette) that includes a first sheet and a second sheet, which may be made from a flexible material, and may be superimposed on each other and joined. A flow path may be formed between the first sheet and the second sheet, and a detection channel portion may be disposed (e.g., midway) along the flow path, where the detection channel portion includes a first bulging portion that may be configured to be wider in a planar direction than the flow path and to bulge in a thickness direction from the first sheet. A second bulging portion may be formed on a portion of the second sheet that faces the first bulging portion. The second bulging portion may be configured to bulge in a thickness direction from the second sheet. The flow path cassette may include a plate member disposed on the second bulging portion, and a deformation preventative member joined to the first bulging portion. The deformation prevention member may include a material that is harder than the first sheet and may be configured to prevent deformation of the first bulging portion and/or to prevent the displacement or deformation of the detection channel portion, which allows both positive and negative pressures acting on, or in, the flow paths to be measured.

Various aspects of the present disclosure relate to a cleaning kit that includes a flow path cassette including integrated flow paths and a plurality of bags and tubes connected to the flow path cassette. The flow path cassette includes first and second sheets that include a flexible material. The first and second sheets may be superimposed (i.e., disposed parallel) and joined in one or more areas or portions. A plurality of flow paths may be disposed between the first sheet and the second sheet, and a detection channel portion may be disposed (e.g., midway) along the flow paths. The detection channel portion may include a first bulging portion that may be configured to be wider in a planar direction than the flow path and to bulge in a thickness direction from the first sheet, a second bulging portion formed on a portion of the second sheet that faces the first bulging portion and may be configured to bulge in a thickness direction from the second sheet, a plate member disposed on the second bulging portion, and a deformation preventative member made of a material that is harder than the first sheet and joined to the outer surface of the first bulging portion of the first sheet. The deformation preventative member may be configured to prevent deformation of the first bulging portion and/or to prevent the displacement or deformation of the detection channel portion, which allows both positive and negative pressures acting on, or in, the flow paths to be measured.

Various aspects of the present disclosure relate to a cell cleaning system equipped with a cleaning kit that includes a flow path cassette including integrated flow paths and a plurality of bags and tubes connected to the flow path cassette. The flow path cassette includes first and second sheets that include a flexible material. The first and second sheets may be superimposed and joined in one or more areas or portions. A plurality of flow paths may be disposed between the first sheet and the second sheet, and a detection channel portion may be disposed (e.g., midway) along the flow paths. The detection channel portion may include a first bulging portion that may be configured to be wider in a planar direction than the flow path and to bulge in a thickness direction from the first sheet, a second bulging portion formed on a portion of the second sheet that faces the first bulging portion and may be configured to bulge in a thickness direction from the second sheet, a plate member disposed on the second bulging portion, and a deformation preventative member made of a material that is harder than the first sheet and joined to the outer surface of the first bulging portion of the first sheet. The deformation preventative member may be configured to prevent deformation of the first bulging portion and/or to prevent the displacement or deformation of the detection channel portion, which allows both positive and negative pressures acting on, or in, the flow paths to be measured.

### DRAWINGS

FIG. 1 illustrates a cell cleaning system including a cleaning kit having a flow path cassette in accordance with various aspects of the present disclosure.

FIG. 2 is an exploded perspective view of the flow path cassette in accordance with various aspects of the present disclosure;

FIG. 3 is an enlarged partial, perspective view of a cassette main body and a frame in accordance with various aspects of the present disclosure;

FIG. 4 is an explanatory diagram illustrating liquid paths of the cleaning kit and flow paths of the cassette main body in accordance with various aspects of the present disclosure;

FIG. 5 is a partial cross-sectional view showing a pressure detecting unit of the cell cleaning system in accordance with various aspects of the present disclosure;

FIG. 6A is a top-down view of a deformation preventative member of the flow path cassette in accordance with various aspects of the present disclosure;

FIG. 6B is a cross-sectional view of the deformation preventative member of the flow path cassette in accordance with various aspects of the present disclosure;

FIG. 7A is a cross-sectional view illustrating a first step for manufacturing the deformation preventative member of the flow path cassette in accordance with various aspects of the present disclosure;

FIG. 7B is a cross-sectional view illustrating a second step for manufacturing the deformation preventative member of the flow path cassette in accordance with various aspects of the present disclosure;

FIG. 7C is a cross-sectional view illustrating a third step for manufacturing the deformation preventative member of the flow path cassette in accordance with various aspects of the present disclosure;

FIG. 8 is a schematic illustrating operations of the cell cleaning system in accordance with various aspects of the present disclosure;

FIG. 9 is a schematic illustrating operations of the cell cleaning system in accordance with various aspects of the present disclosure;

FIG. 10 is a schematic illustrating operations of the cell cleaning system in accordance with various aspects of the present disclosure; and

FIG. 11 is a schematic illustrating operations of the cell cleaning system in accordance with various aspects of the present disclosure.

### DETAILED DESCRIPTION

Certain embodiments of the present invention will be presented and described in detail below with reference to the accompanying drawings.

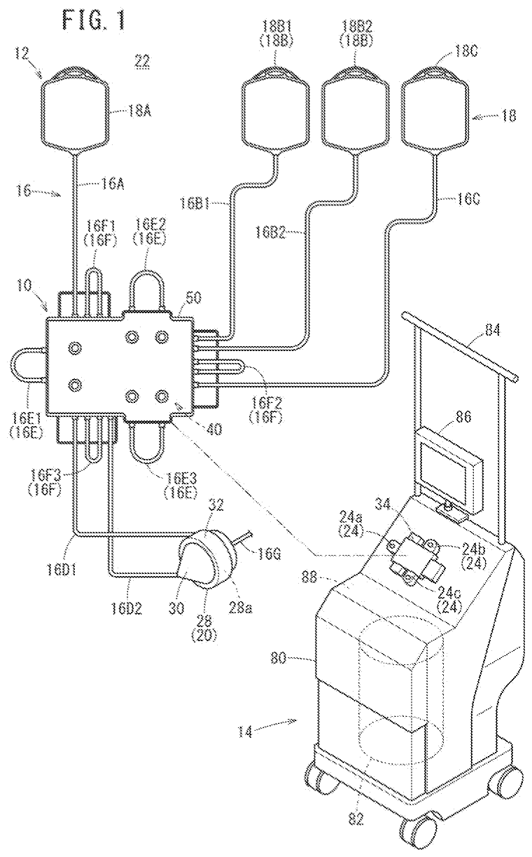

As illustrated in FIG. 1, a cell cleaning device 14 is configured to receive a cleaning kit 12 that includes a flow path cassette 10. The flow path cassette 10 includes a plurality of flow paths (also referred to as paths) configured to be integrated with certain flow paths of the cleaning kit 12 and through which liquids, such as a medium used of culturing cells and/or a liquid such as a cleaning solution for treating cultured cells, may be capable of flowing (e.g., circulated).

In addition to the flow path cassette 10, the cleaning kit 12 includes a plurality of tubes 16, a plurality of bags 18 (e.g., medical bags), and a treatment unit 20 in which processing may be performed in the cell cleaning device 14. The cleaning kit 12 is configured such that liquids contained in the different bags 18 of the plurality can be processed by the treatment unit 20 and can flow through the flow path cassette 10, and also through each of the tubes 16 under the operation of the cell cleaning device 14.

The cleaning kit 12 may be configured as a disposable type of kit, and may include a culture having cells of a living body and/or a medium or a preservative solution in the form of a liquid flowing through the interior thereof and/or a cleaning solution for cleaning the cells. The cleaning kit 12 and the cell cleaning device 14 may include a cell cleaning system 22 that can be applied to a cleaning process, which may be one step of a regenerative medicine process. Using the cleaning solution, the cell cleaning system 22 may perform the cleaning process for removing the culture medium and the like from the culture having the cultured cells, thereby increasing the cell concentration.

The cells of a living body are not particularly limited, and may include, for example, cells (e.g., T cells and the like) contained in blood and/or stem cells (e.g., ES cells, iPS cells, mesenchymal stem cells, and the like). The cleaning solution may be selected appropriately depending on the cells of the living body, and may in certain variations, include, for example, a buffer solution such as PBS (Phosphate Buffered Salts), TBS (Tris-Buffered Saline), or physiological saline or the like can be applied thereto.

The plurality of bags 18 may include a culture bag 18A in which the culture may be accommodated, one or more cleaning solution bags 18B (e.g., a first cleaning solution bag 18B1 and a second cleaning solution bag 18B2) in which the cleaning solution may be accommodated, and a product bag 18C for accommodating the cells that have been cleaned.

In a culturing step of a regenerative medicine process, the culture bag 18A may store cells which have been cultured. The cleaning solution bag 18B may be assembled into the cleaning kit 12 in a state with the cleaning solution stored therein, sealed using a sterile bonding apparatus or the like. Alternatively, the cleaning solution bag 18B may be assembled beforehand in the cleaning kit 12 in a state in which the cleaning solution may be stored and sealed therein, and the seal may be released at the time of being set. The product bag 18C may be connected in an empty state before the cleaning process is performed, and the cells (including the cleaning solution) which have been concentrated accompanying the cleaning process may be stored to become the product. A preservative solution or the like may also be stored beforehand in the product bag 18C.

The plurality of tubes 16 of the cleaning kit 12 may include a culture tube 16A that connects the culture bag 18A and the flow path cassette 10, a first cleaning solution tube 16B1 that connects the first cleaning solution bag 18B1 and the flow path cassette 10, a second cleaning solution tube 16B2 may connect the second cleaning solution bag 18B2 and the flow path cassette 10, a product tube 16C may connect the product bag 18C and the flow path cassette 10, and two tubes 16, including a first treatment tube 16D1 and a second treatment tube 16D2, may connect the treatment unit 20 and the flow path cassette 10. Further, the plurality of tubes 16 may also include closed tubes 16E, 16F that protrude from the flow path cassette 10 and may be folded back and connected to the flow path cassette 10 again. For example, as illustrated, there may be a plurality of pump tubes 16E that may be set in a plurality of pumps 24 of the cell cleaning device 14, and a plurality of clamp tubes 16F that may be set within clamps 26 for opening and closing the paths of the cleaning kit 12. In certain variations, as illustrated, the plurality of tubes 16 may include three pump tubes 16E, three clamp tubes 16F. Further, the plurality of tubes 16 may also include a disposal tube 16G that communicates with the exterior of the cleaning kit 12 (for example, with a non-illustrated disposal unit of the cell cleaning device 14) without intervening from the treatment unit 20 to the flow path cassette 10.

Connecting structures (not shown) that can be connected together mutually may be provided on the culture tube 16A and the culture bag 18A, the first cleaning solution tube 16B1 and the first cleaning solution bag 18B1, and/or the second cleaning solution tube 16B2 and the second cleaning solution bag 18B2. The culture bag 18A, the first cleaning solution bag 18B1, and/or the second cleaning solution bag 18B2 may be connected to the culture tube 16A, the first cleaning solution tube 16B1, and/or the second cleaning solution tube 16B2 through the connecting structures prior to implementing the cleaning process. Consequently, the culture may be able to flow out to the flow path of the culture tube 16A, and the cleaning solution may be able to flow out to the first cleaning solution tube 16B1 and the second cleaning solution tube 16B2.

The treatment unit 20 of the cleaning kit 12 may include a treatment case 28 in which the culture and the cleaning solution may be temporarily accommodated, and by applying a centrifugal force from the cell cleaning device 14, the culture, which may be accommodated in an internal space 28a, may be centrifugally separated into the cells and the culture medium. The treatment case 28 may be formed in a hollow three-dimensional shape formed by joining a conical portion 30 and a cylindrical portion 32 in an axial direction. In a set state of the cell cleaning device 14, in the treatment case 28, an apex of the conical portion 30 may be arranged on a side remote from the center of centrifugation, and a bottom portion of the cylindrical portion 32 may be arranged on a side proximate to the center of centrifugation.

When a centrifugal force is applied thereto, the treatment case 28 may move cells having a high specific gravity outward in the centrifugal direction within the tapered internal space 28a and the culture medium having a low specific gravity inward in the centrifugal direction. The first treatment tube 16D1 may be connected to a side of the cylindrical portion 32 of the treatment case 28, whereas the second treatment tube 16D2 may be connected to the apex of the conical portion 30. Further, the disposal tube 16G may be connected to the bottom of the treatment case 28 in order to discard the culture medium that was separated by centrifugation.

In addition, by joining the plurality of tubes 16 in advance, the flow path cassette 10 may become a relay unit of the cleaning kit 12, which circulates the culture or the cleaning solution in each of the respective bags 18 to other bags 18 or to the treatment unit 20. When the cleaning kit 12 is set in the cell cleaning device 14, the flow path cassette 10 may be attached to a cassette setting location 34 of the cell cleaning device 14, which can simplify the wiring operation of the tubes 16 in the cleaning process.

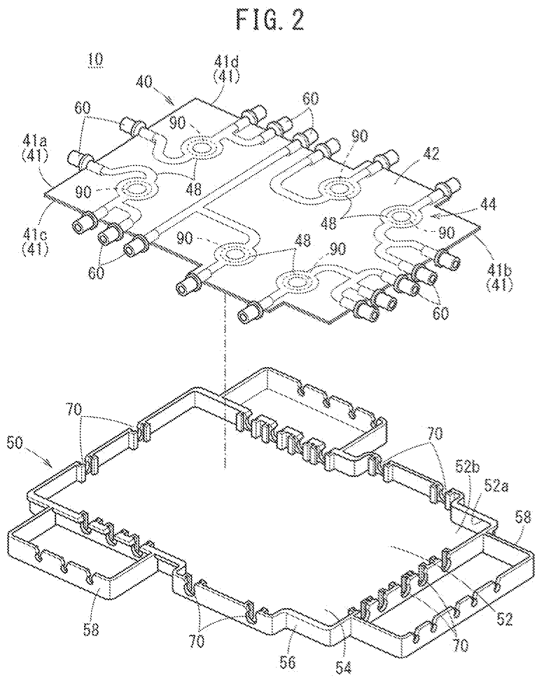

As illustrated in FIG. 2, the flow path cassette 10 includes a soft cassette main body 40 and the plurality of tubes 16 coupled thereto. For example, the tubs 16 may be directly connected to the soft cassette main body 40. The flow path cassette 10 may also include a rigid frame 50 that may be configured to receive the cassette main body 40. The rigid frame 50 may be fixed to the cell cleaning device 14.

The cassette main body 40 may include a flexible, thin sheet having a substantially rectangular shape. For example, in certain variations, the cassette main body 40 may include a resin layer 42 including two or more resin sheets stacked and joined together at one or more portions or pieces. The resin material constituting the resin sheets 42 is not particularly limited, insofar as it possesses flexibility that may be capable of being deformed by the pressure of the liquids. For example, in certain variations, the resin sheets 42 may include a vinyl chloride resin, a polyolefin resin, and/or a polyurethane resin.

The cassette main body 40 may also include a plurality of flow paths 44 disposed on a first (or inner) side of the cassette main body 40. The flow paths 44 may be fusion bonded to the resin layer 42. For example, in certain variations, the flow paths 44 may be deposed between a first resin sheet of the two or more resin sheets and a second resin sheet of the two or more resin sheets and the first and second resin sheets may be bonded in the areas surrounding the flow paths 44, such that the flow paths 44 define protrusions in cassette main body 40. A plurality of connectors 60 for connecting the plurality of tubes 16 may be provided on outer edges 41 of the cassette main body 40.

The frame 50 may include a resin material that is harder than that of the cassette main body 40. The frame 50 may be formed to define a recess that defines an accommodation chamber 52 which may be configured to receive the cassette main body 40. The rigid material of the frame 50 is not limited to any particular material. In certain variations, however, the rigid material may include a thermoplastic resin material, such as polypropylene, polycarbonate, polyamide, polysulfone, polyacrylate, and/or methacrylate-butylene-styrene copolymer.

The frame 50 may include a cover portion 54 having a same general shape as the cassette main body 40 and being slightly larger than the cassette main body 40 so as to be configured to receive the cassette main body 40. For example, in certain variations, the frame 50 may have a substantially rectangular shape. The frame 50 may include a side portion 56 that protrudes substantially perpendicularly from an outer periphery of the cover portion 54. In certain variations, as illustrated, the side portion 56 may extend around an entire outer periphery of the cover portion 54.

The accommodation chamber 52 may be opened through an opening 52a that is surrounded by the side portions 56 on an opposite side of the cover portion 54, such that one surface of the cassette main body 40 may be exposed. In other words, the accommodation chamber 52 may be defined by a concave shape that is formed by the side portions 56 and a bottom portion 52b of the accommodation chamber 52, and when received, the surface of the cassette main body 40 may be covered by the bottom portion 52b of the accommodation chamber 52. In the accommodation chamber 52, the bottom portion 52b may include the cover portion 54, and the bottom portion 52b may be opened by the frame without the presence of the cover portion 54.

Engaging portions 70 in which the respective connectors 60 may be retained are provided in the side portions 56 at positions corresponding to each of the connectors 60 of the cassette main body 40. The connectors 60 and the engaging portions 70 may define engagement mechanisms 68 for coupling the frame 50 and the cassette main body 40.

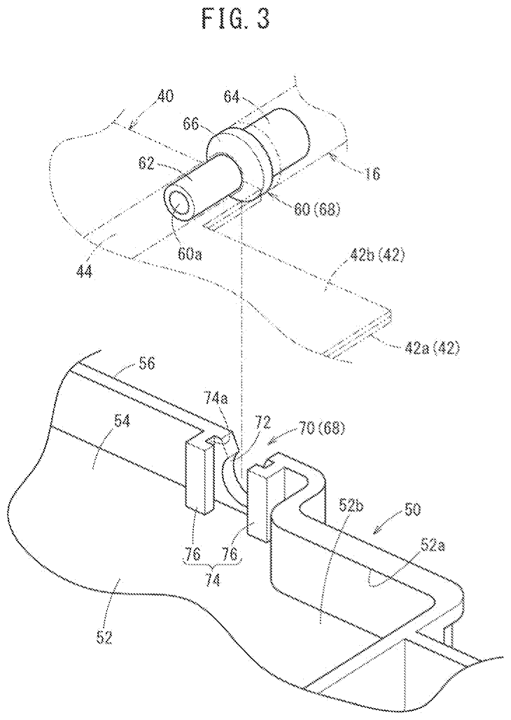

As illustrated in FIG. 3, each of the connectors 60 of the cassette main body 40 may include a first cylindrical part 62 that may be sealed to the cassette main body 40, a second cylindrical part 64 that may be connected to the tube 16, and a flange 66 that protrudes radially outward between the first cylindrical part 62 and the second cylindrical part 64. A communication hole 60a that penetrates through the first cylindrical part 62, the second cylindrical part 64, and the flange 66 may be formed in the axial center of the connector 60.

At a time of sealing, that is, when the two resin sheets 42 of the cassette main body 40 may be placed together and sealed, the first cylindrical parts 62 may be fusion bonded to the cassette main body 40, in a state in which the communication holes 60a communicate with the flow paths 44 of the flow path cassette 10. An outer peripheral surface of the first cylindrical parts 62 may be formed with a smaller diameter than the second cylindrical parts 64 in order to correspond with the flow paths 44 of the flow path cassette 10. Further, the second cylindrical parts 64 may be inserted inside the tubes 16, and may be firmly fixed to the tubes 16 by an appropriate fixing means. The flanges 66 may have a predetermined thickness in the axial direction of the connectors 60. For example, the flanges 66 may be formed in a ring shape that encircles the entire outer peripheral surface of the connectors 60.

The engaging portions 70 of the frame 50 may include engagement recesses 72 in which the side portions 56 are absence. The engaging portions 70 may also include movement limiters 74 that protrude from the side portions 56 toward the inner side of the frame 50 in close proximity to the engagement recesses 72. The engagement recesses 72 may be opened in the same direction as the opening 52a of the frame 50, and may be formed in arcuate shapes (e.g., C-shapes) which may be capable of accommodating the tubes 16 connected to the connectors 60 (e.g., second cylindrical parts 64). The engagement recesses 72 may be set to a size that enables them to be firmly fitted with respect to the accommodated tubes 16 and the connectors 60.

Each of the movement limiters 74 may include a pair of hook portions 76 which protrude inwardly from the inner surface of the side portions 56, and may be bent in perpendicular directions and in directions to approach mutually toward each other. The movement limiters 74 may allow the flanges 66 of the connectors 60 to be accommodated in fixed spaces 74a formed between the movement limiters 74 and the side portions 56.

Movement of the connectors 60 in the axial direction may be restricted by disposing the flanges 66 in the fixed spaces 74a. Further, the connectors 60 may be accommodated in the engagement recesses 72 together with the tubes 16, whereby the connectors 60 become engaged with the engaging portions 70 (side portions 56) at an appropriate engagement force, and the connectors 60 may be prevented from slipping out from the frame 50. By the respective connectors 60 of the cassette main body 40 being retained by the respective engaging portions 70 of the frame 50, the flow path cassette 10 may be placed in a state in which the cassette main body 40 and the frame 50 may be integrated, which is understood to be a state in which the cassette main body 40 and the frame 50 may be handled together, collectively.

Returning to FIG. 2, the aforementioned engagement mechanisms 68 may be disposed respectively on four sides of the substantially rectangular shaped flow path cassette 10. The cassette main body 40 may be equipped with the connectors 60 at each of four outer edges 41, whereas the frame 50 may also be equipped with the engaging portions 70 at each of four side portions 56. Consequently, the frame 50 may be capable of retaining the sheet-shaped cassette main body 40 in a stretched state, and can suitably cause the flow paths 44 to be extended along a planar direction.

In addition, the flow path cassette 10 may be attached to the cell cleaning device 14 in a state in which the cassette main body 40 and the frame 50 may be integrated, and with the surface opposite to the illustrated surface being held in a reverse manner. Accordingly, in a mounted state, one surface of the cassette main body 40 looking out from the opening 52a of the frame 50 may face toward an upper surface of the cell cleaning device 14, and the cover portion 54 may cover the entirety of the cassette main body 40. Stated otherwise, in the mounted state, the upper and lower sides of the cassette main body 40 in FIG. 2 are inverted.

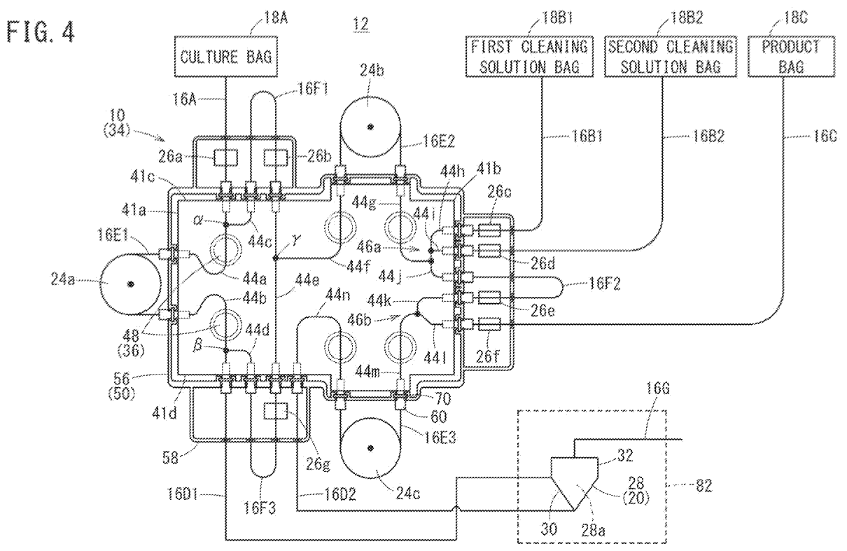

FIG. 4 illustrates example paths for liquids in the cleaning kit 12 together with the flow paths 44 of the flow path cassette 10. The flow path cassette 10 as shown in FIG. 4 is provided in plan view in a state in which the flow path cassette 10 is attached to the cell cleaning device 14, and in order to facilitate description, is illustrated in a state with the cover portion 54 of the frame 50 omitted. The outer edges 41 of the cassette main body 40 include a first short side 41a, a second short side 41b, a first long side 41c, and a second long side 41d.

The culture tube 16A may be joined to the culture bag 18A and may be connected to the first long side 41c. The first cleaning solution tube 16B1 may be joined to the first cleaning solution bag 18B1, the second cleaning solution tube 16B2 may be joined to the second cleaning solution bag 18B2, and the product tube 16C may be joined to the product bag 18C. The first cleaning solution tube 16B1, the second cleaning solution tube 16B2, and the product tube 16C may be connected to the second short side 41b. The first and second treatment tubes 16D1 and 16D2 may be joined to the treatment unit 20 and may be connected to the second long side 41d.

In the cell cleaning system 22, when the cell cleaning device 14 and the cleaning kit 12 may be set, the three pumps 24 may be arranged at positions in proximity to the sides of the flow path cassette 10. For example, in the set state, the cassette setting location 34 may include a first pump 24a disposed in proximity to the first short side 41a, a second pump 24b disposed in proximity to the first long side 41c, and a third pump 24c disposed in proximity to the second long side 41d. Therefore, the respective pump tubes 16E of the cleaning kit 12 may include a first pump tube 16E1 connected to the first short side 41a, a second pump tube 16E2 connected to the first long side 41c, and a third pump tube 16E3 connected to the second long side 41d.

The first to third pump tubes 16E1 to 16E3 may be arranged in a manner so that the portions that may be folded back in an arcuate shape may be wrapped around circular shaped wound portions of the first to third pumps 24a to 24c. By being rotated around the respective wrapped around pump tubes 16E, the first to third pumps 24a to 24c may apply a fluid force to the liquids in each of the pump tubes 16E.

In the cell cleaning system 22, when the cleaning kit 12 is set with respect to the cell cleaning device 14, a plurality of the clamps 26 may be arranged at positions in proximity to the sides of the flow path cassette 10. For example, in the set state, the cassette setting location 34 may include first and second clamps 26a and 26b disposed in proximity to the first long side 41c, third to sixth clamps 26c to 26f disposed in proximity to the second short side 41b, and a seventh clamp 26g disposed in proximity to the second long side 41d.

The culture tube 16A may be arranged on a first clamp 26a, the first cleaning solution tube 16B1 may be arranged on a third clamp 26c, the second cleaning solution tube 16B2 may be arranged on a fourth clamp 26d, and the product tube 16C may be arranged on a sixth clamp 26f. Further, the clamp tubes 16F may include a first clamp tube 16F1 connected to the first long side 41c and arranged on the second clamp 26b, a second clamp tube 16F2 connected to the second short side 41b and arranged on the fifth clamp 26e, and a third clamp tube 16F3 connected to the second long side 41d and arranged on the seventh clamp 26g.

To reliably carry out opening and closing of the flow paths 44 of the tubes 16 using the clamps 26, the frame 50 of the flow path cassette 10 may include a plurality of retaining frames 58 that can extend from the side portions 56 and retain the tubes 16. The respective retaining frames 58 may be provided on the side portions 56 corresponding to the first and second long sides 41c and 41d and the second short side 41b, and retain outer peripheries of the tubes 16 that may be separated by a predetermined distance from the side portions 56.

The flow paths 44 of the cassette main body 40 may communicate with the respective tubes 16 via each of the connectors 60. For example, The flow paths 44 may include a first path 44a that places one end (i.e., a first end) of the first pump tube 16E1 in communication with the culture tube 16A, a second path 44b that places the other end (i.e., a second end distal to the first end) of the first pump tube 16E1 in communication with the first treatment tube 16D1, a third path 44c that places a connection point alpha of the first path 44a in communication with one end (i.e., a first end) of the first clamp tube 16F1, a fourth path 44d that places a connection point beta of the second path 44b in communication with one end (i.e., a first end) of the third clamp tube 16F3, a fifth path 44e that places the other end (i.e., a second end) of the third clamp tube 16F3 in communication with the other end (i.e., a second end distal to the first end) of the first clamp tube 16F1, a sixth path 44f that places a connection point gamma of the fifth path 44e in communication with one end (i.e., a first end) of the second pump tube 16E2, a seventh path 44g that places a first flow path switching unit 46a provided on the second short side 41b in communication with the other end (i.e., a second end distal to the first end) of the second pump tube 16E2, an eighth path 44h that places the first flow path switching unit 46a in communication with the first cleaning solution tube 16B1, a ninth path 44i that places the first flow path switching unit 46a in communication with the second cleaning solution tube 16B2, a tenth path 44j that places the first flow path switching unit 46a in communication with one end (i.e., a first end) of the second clamp tube 16F2, an eleventh path 44k that places a second flow path switching unit 46b provided on the second short side 41b in communication with the other end (i.e., a second end distal to the first end) of the second clamp tube 16F2, a twelfth path 44l that places the second flow path switching unit 46b in communication with the product tube 16C, a thirteenth path 44m that places the second flow path switching unit 46b in communication with one end (i.e., a first end) of the third pump tube 16E3, and/or a fourteenth path 44n that places the second treatment tube 16D2 in communication with the other end (i.e., a second end distal to the first end) of the third pump tube 16E3.

At the connection points alpha, beta, and gamma, at the first flow path switching unit 46a, and at the second flow path switching unit 46b, the connected flow paths 44 may communicate with each other, and the liquid in one of the flow paths 44 may be allowed to flow freely to the other of the flow paths 44. It should be noted that, although the first flow path switching unit 46a has two connection points for connecting the seventh to tenth paths 44g to 44j, to facilitate explanation, the connection points may be considered together as a single entity.

The cassette main body 40 may include one or more detection channel portions 48 for detecting the pressure of the flow paths 44. The one or more detection channel portions 48 may be disposed along one or more of the flow paths 44. For example, the respective detection channel portions 48 may be disposed at about a halfway or midway point along each flow path 44. The detection channel portions 48 may be capable of detecting the pressure of the liquid flowing therein using a deformation preventative member 90, as later described, that may be provided on the frame 50, and by load cells 100 of the cell cleaning device 14.

Returning to FIG. 1, the cell cleaning device 14 to which the cleaning kit 12 may be attached may include a box-shaped device main body 80, a rotor 82 that may be rotatably accommodated inside the device main body 80, and a stand 84 on which the respective bags 18 of the cleaning kit 12 may be held. The cell cleaning device 14 may also include a display operation unit 86 that provides a user interference that displays information. The cell cleaning device 14 may also include a control unit 88 that may be used alone or in connection with the display operation unit 86 to perform and control operations of the cell cleaning system 22. The cell cleaning device 14 may also include a cassette setting location 34 on an outer surfaces of the device main body 80 that may be configured to receive the flow path cassette 10.

The rotor 82 may have a generally cylindrical shape. The rotor 82 may be disposed below the cassette setting location 34. The rotor 82 may be rotated about an axis by a non-illustrated rotary drive source that may also be provided in the device main body 80. By undergoing rotation in a state with the treatment unit 20 (treatment case 28) of the cleaning kit 12 being accommodated therein, the rotor 82 may apply a centrifugal force to the liquid that flows into the treatment unit 20.

The cassette setting location 34 may include a frame-like structure, which may be formed on the upper inclined surface of the device main body 80, and may be constituted in a manner so that, when the flow path cassette 10 may be fitted on the inner side of the frame-like structure, the frame 50 may be locked by non-illustrated hooks.

The cassette setting location 34 may be equipped with the first to third pumps 24a to 24c and the first to seventh clamps 26a to 26g on the outer peripheral side of the frame-like structure, together with enabling placement thereon of the retaining frames 58 of the flow path cassette 10. Accompanying the attachment of the flow path cassette 10 to the cassette setting location 34, under an operation of the user, the plurality of pumps 24 and the clamps 26 may be arranged with appropriate tubes 16 of the cleaning kit 12.

The cell cleaning system 22 may detect the pressure (i.e., pressure state) of the liquid that flows in the predetermined flow paths 44 of the flow path cassette 10. For example, the cell cleaning system 22 may include one or more pressure detecting units 36 in each of the first path 44a, the second path 44b, the sixth path 44f, the seventh path 44g, the thirteenth path 44m, and the fourteenth path 44n inside the flow path cassette 10. In other words, each of the pressure detecting units 36 may be provided respectively on an upstream side and a downstream side of the first to third pumps 24a to 24c. In addition, on the basis of the pressures (i.e., differential pressures) of the respective pressure detecting units 36, the control unit 88 of the cell cleaning device 14 may calculate the flow rates at times that the first to third pumps 24a to 24c may be driven, and the calculated flow rates may be fed back to the control of the first to third pumps 24a to 24c.

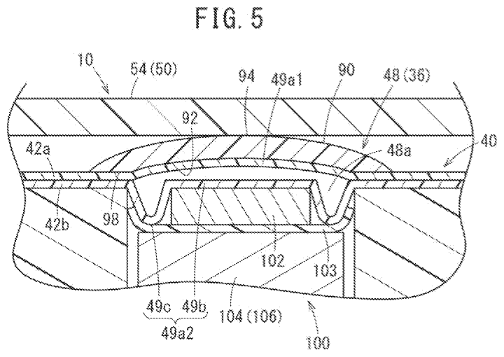

As illustrated in FIG. 5, in a set state (i.e., where the cell cleaning device 14 receives the flow path cassette 10), each of the pressure detecting units 36 define a detection channel portion 48 and the deformation preventative member 90 of the flow path cassette 10, and the load cell 100 of the cell cleaning device 14. The respective pressure detecting units 36 may grip (e.g., sandwich) the detection channel portions 48 using the deformation preventative members 90 and the load cells 100, and can detect the loads (e.g., pressures) of the detection channel portions 48 when the liquid flows through the detection channel portions 48.

In a plan view of the cassette main body 40, the detection channel portions 48 may be formed to be wider in a planar direction than the consecutively disposed flow paths 44. For example, in certain variations, the detection channel portions 48 may be formed so as to extend in a substantially perfect circular shape. As illustrated, each of the detection channel portions 48 may include a first bulging portion 49a1 and a second bulging portion 49a2 whose cross-sectional shapes bulge in the thickness direction away from a first sheet 42a and a second sheet 42b, respectively. That is, the first bulging portion 49a1 may bulge in a first direction relative to a major axis of the flow path cassette 10, and the second bulging portion 49a2 may bulge in a second direction relative to the major axis of the flow path cassette 10 and away from the first direction. The first bulging portion 49a1 of the first sheet 42a may bulge outward in a mountain-like manner (i.e., concave relative to a major axis of the first sheet 42a) so that a central portion (i.e., apex) thereof may be the highest. Further, the second bulging portion 49a2 of the second sheet 42b may include a flat or center portion 49b at the center thereof and one or more protrusions 49c that bulge away from the flat portion 49b and in the second direction. The flat portion 49b may be a portion that may be aligned with a plate member 102. In certain variations, the plate member 102 may be joined to or contacted with a plate member 102.

Between the first bulging portions 49a1 and the second bulging portions 49a2, flow chambers 48a may be formed through which the liquid flows in the detection channel portions 48. The flow chambers 48a may communicate with the flow paths 44.

The deformation preventative members 90 may be attached beforehand to the first bulging portions 49a1 on the side of the frame 50 (cover portion 54). The plate members 102 may be joined to the central flat portions 49b of the second bulging portions 49a2. The plate members 102, for example, may be made from a ferromagnetic material, such as iron or ferritic stainless steel, which may be capable of being attracted to a magnet (e.g., magnetophilic metal), and may be joined to the second sheet 42b by fusion bonding or using an adhesive. The plate members 102 may be formed in a shape (e.g., circular shape) that coincides substantially with the shape of the flat portions 49b, and in the case that liquid may not flow through the detection channel portions 48 and almost no load may be received, the plate members 102 may cooperate with the deformation preventative members 90 to make the interval between the first bulging portions 49a1 and the second bulging portions 49a2 substantially constant. In the set state, the load cells 100 may be disposed adjacent to the plate members 102. The plate members 102 may be displaced depending on the internal pressure of the detection channel portions 48, and may cause the attractive force that acts on magnets 104 of the load cells 100 to change.

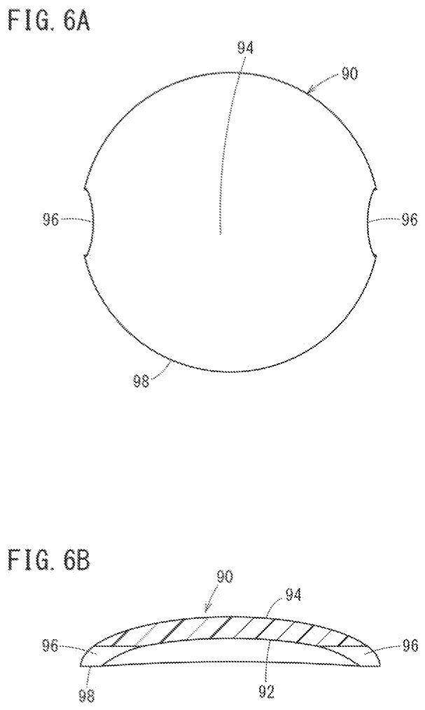

As illustrated in FIGS. 6A and 6B, each of the deformation preventative members 90 may be formed with a circular shape as viewed in plan, and may be formed with a smoothly curved dome shape so as to bulge out from an outer peripheral portion 98 toward a central top portion 94 thereof. On the inner side of each of the deformation preventative members 90, a concave portion 92 may be formed which may be curved along the first bulging portion 49a1 of the first sheet 42a. The entire area of the first bulging portion 49a1 may be firmly joined to the concave portion 92 without any gaps. Further, as illustrated in FIG. 6A, notches 96 that enable the flow paths 44 to pass therethrough may be formed at one end and another end of each of the deformation preventative members 90.

The deformation preventative members 90 may be configured to be harder than the first sheet 42a, and may be fixed to the detection channel portions 48 using an appropriate fixing means. The material constituting the deformation preventative members 90 is not particularly limited. For example, the deformation preventative members 90 may include resin materials like those noted for frame 50 and/or certain metal material.

As illustrated in FIG. 5, when a positive pressure is applied to the flow chamber 48a, by the top portion 94 abutting against the cover portion 54, the deformation preventative member 90 may prevent the displacement (i.e., escape) of the first sheet 42a. As a result, the distance between the load cells 100 and the detection channel portions 48 may be maintained at an appropriate distance and an accurate pressure measurement may be performed.

Further, when a negative pressure is applied to the flow chamber 48a, the outer peripheral portion 98 of the deformation preventative member 90 may abut against the cassette setting location 34 with the first and second sheets 42a and 42b intervening therebetween. In addition, by preventing the first bulging portion 49a1 which may be joined to the concave portion 92 from being crushed, measurement of a negative pressure is made possible.

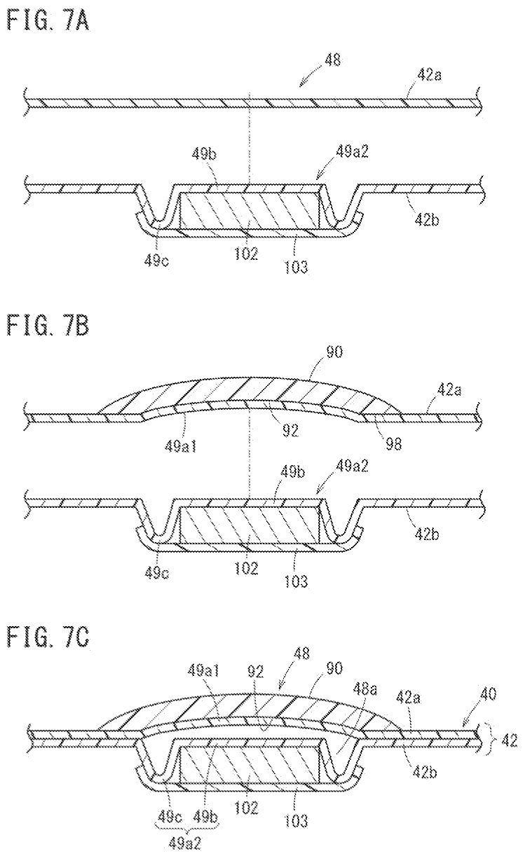

In various aspects, the present disclosure provides methods for forming detection channel portions (like the detection channel portions 48) during the manufacturing of flow path cassettes, like the flow path cassette 10. For example, in certain variations, an example method may include, as illustrated in FIG. 7A, preparing the first sheet 42a and the second sheet 42b. For example, preparing the second sheet 42b may include heating a precursor sheet while pressing the precursor sheet against a mold having a cavity, corresponding to the second bulging portions 49a2. Preparing the second sheet 42b may also include disposing and aligning the plate members 102 with the central portion (or apex) of the second bulging portions 49a2. Further still, preparing the second sheet 42b may also include covering an exposed surface of the plate members 102 with covering sheets 103 and joining the covering sheets 103 with the second sheet. The covering sheets 103 may be joined to the second sheet 42b using fusion bonding and/or an adhesive.

The method for preparing the detection channel portions 48 of the flow path cassette 10 may also include, as illustrated in FIG. 7B, joining the deformation preventative members 90 to the first sheet 42a. The deformation preventative members 90 may be joined to the first sheet 42a by pressing the first sheet 42a against the deformation preventative members 90 using compressed air or the like. In this manner, portions of the first sheet 42a may bulge along the deformation preventative members 90 to form the first bulging portions 49a1. Once joined, the method the deformation preventative members 90 and the first bulging portions 49a1 may be joined using known method, such as high frequency fusion bonding, heat welding, and/or adhesion.

The method for preparing the detection channel portions 48 of the flow path cassette 10 may also include, as illustrated in FIG. 7C, joining together the non-bulging portions of the first sheet 42a and the second sheet 42b, simultaneously forming the flow paths 44 and the joining the connectors 60.

In various aspects, the present disclosure provides a load measurement method to be applied to each of the load cells 100 of the cell cleaning device 14. For example, as illustrated in FIG. 5, each of the load cells 100 may be equipped with a magnet 104 disposed at the cassette setting location 34, and a load detecting unit 106 having the magnet 104 mounted on an upper part thereof. The magnet 104 may be maintained in a state of being attracted to the plate member 102 of the second sheet 42b. When the plate members 102 may be displaced upward due to a negative pressure acting on the detection channel portions 48, in following relation to such displacement, the magnets 104 apply a negative load corresponding the negative pressure to the load detection units 106.

The plate members 102 may be displaced when the internal pressures of the detection channel portions 48 of the cassette main body 40 may be changed, causing a load change to occur in the magnets 104. The load detecting units 106 may be configured to detect changes in the loads of the magnets 104 in accordance with the displacement of the plate members 102, and to transmit load detection signals of the detection channel portions 48 to the control unit 88 of the cell cleaning device 14.

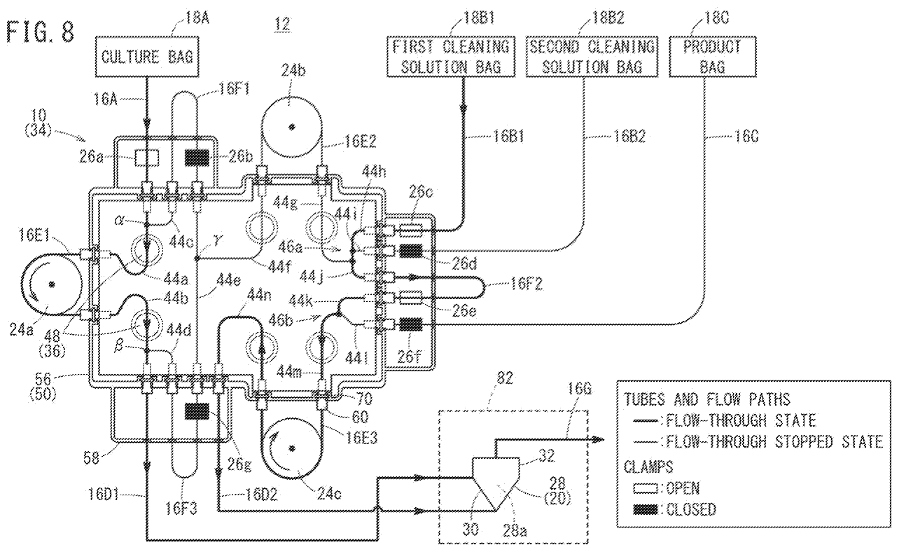

FIGS. 8 to 11 illustrate example operations for cell processes using cell cleaning systems, like the cell cleaning system 22 illustrated in FIG. 1. For example, FIGS. 8 to 11 illustrate example operations of the tubes 16 and of the flow paths 44 of the flow path cassette 10.

In an example cleaning process using the cell cleaning system 22, a user or operator may contact the cleaning kit 12 including the flow path cassette 10 with the cell cleaning device 14. In doing so, the cleaning kit 12 may be arranged with the pumps 24 and the clamps 26 of the cell cleaning device 14, as illustrated in FIG. 4.

After such setting is completed, the cleaning process may include a priming step that includes operating one or more of the first to third pumps 24a to 24c and supplying a cleaning solution of the first or second cleaning solution bags 18B1 and 18B2 into the flow paths 44 of the flow path cassette 10 and also into the internal space 28a of the treatment case 28. That is, the air in the flow paths 44 and the internal space 28a may escape, and the flow paths 44 and the internal space 28a may be filled with the cleaning solution.

Following the priming step, as illustrated in FIG. 8, the cleaning process may include a first culture supplying step. The first culture supplying step may include transferring, for example supplying, the culture in the culture bag 18A to the treatment case 28. In this first culture supplying step, the cell cleaning device 14 may drive the first pump 24a to transfer the culture to the treatment unit 20 by placing in communication a path from the culture bag 18A to the treatment unit 20. For example, the cell cleaning device 14 may open the first clamp 26a, while closing the second clamp 26b and the seventh clamp 26g. As such, the culture in the culture bag 18A can pass through the culture tube 16A, the first path 44a, the first pump tube 16E1, and the second path 44b, so as to flow into the first treatment tube 16D1, whereupon the culture may flow into the internal space 28a from the side of the treatment case 28. At the time that the culture may be transferred, the cell cleaning device 14 may cause the rotor 82 to rotate at a selected speed of rotation (e.g., 3000 rpm) and applies a centrifugal force to the treatment case 28.

The culture supplying step may include supplying the cleaning solution to the treatment case 28, in conjunction with the culture being supplied to the treatment case 28. For example, in the cell cleaning device 14, the third clamp 26c and the fifth clamp 26e may be opened, while on the other hand, the fourth clamp 26d and the sixth clamp 26f may be closed, while the third pump 24c may be driven. As such, the cleaning solution in the first cleaning solution bag 18B1 may pass through the first cleaning solution tube 16B1, the eighth path 44h, the tenth path 44j, the second clamp tube 16F2, the eleventh path 44k, the thirteenth path 44m, the third pump tube 16E3, and the fourteenth path 44n, and may flow into the second treatment tube 16D2, whereupon the cleaning solution may flow into the internal space 28a from the apex of the treatment case 28.

In the culture supplying step, the amount and rate of the culture supplied to the treatment case 28 may be predetermined or preselected. For example, in certain variations, a rate of the culture supplied to the treatment case 28 may be greater than or equal to about 5 mL/min to less than or equal to about 50 mL/min. The amount of the cleaning solution supplied to the treatment case 28 may also be predetermined or preselected. For example, in certain variations, the rate of the cleaning solution supplied to the treatment case 28 may be about 2.5 mL/min. By preventing the cells of the culture inside the treatment case 28 from being strongly pressed outward in the centrifugal direction by the cleaning solution, the cells can be protected. For example, because the specific gravity of the cleaning solution and the culture medium contained within the culture inside the treatment case 28 may be lower than that of the cells, the cleaning solution and the culture medium may be moved to the bottom of the treatment case 28 by the centrifugal force and may flow out to the disposal tube 16G in communication with the treatment case 28.

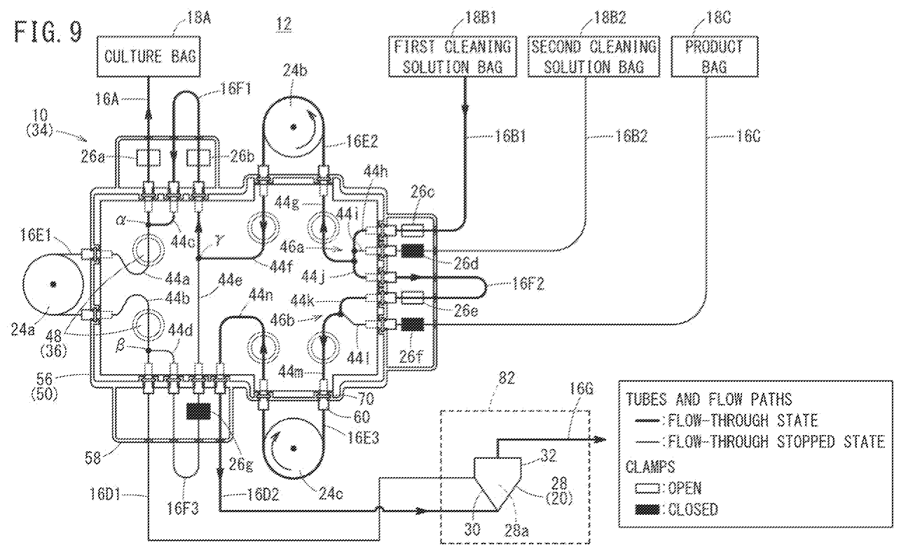

As illustrated in FIG. 9, the cleaning solution may be supplied to the culture bag 18A, and a peeling step may be carried out to peel off cells that may be adhered to the culture bag 18A. During the peeling step, the cell cleaning device 14 places in communication a path from the first cleaning solution bag 18B1 to the culture bag 18A. For example, the cell cleaning device 14 may open the first clamp 26a, the second clamp 26b, and the third clamp 26c, while closing the fourth clamp 26d and the seventh clamp 26g. As such, the cleaning solution in the first cleaning solution bag 18B1 may pass through the first cleaning solution tube 16B1, the eighth path 44h, the seventh path 44g, the second pump tube 16E2, the sixth path 44f, the fifth path 44e, the first clamp tube 16F1, the third path 44c, and the first path 44a, and flow into the culture tube 16A, whereupon the cleaning solution may flow into the culture bag 18A.

In certain variations, simultaneous with the peeling, the cleaning solution may be supplied to the treatment case 28, and further the rotor 82 may be rotated to apply a centrifugal force to the treatment case 28. For example, in the cell cleaning device 14, the third clamp 26c and the fifth clamp 26e may be opened, while the fourth clamp 26d and the sixth clamp 26f may be closed, and the third pump 24c may be driven. As such, inside the treatment case 28, centrifugation of the culture and disposal of the culture medium and the cleaning solution may continue.

In addition, in the cell cleaning system 22, after completion of the peeling step, a second culture supplying step may occur. The second culture supplying step may include the same processes as the first culture supplying step. For example, the cells peeled off in the peeling step may be supplied to the treatment case 28 and the peeled off culture in the treatment case 28 may be subjected to centrifugation. The culture medium may be discarded from the treatment case 28 together with the supplied cleaning solution. As such, the process can be performed without leaving cells inside the culture bag 18A.

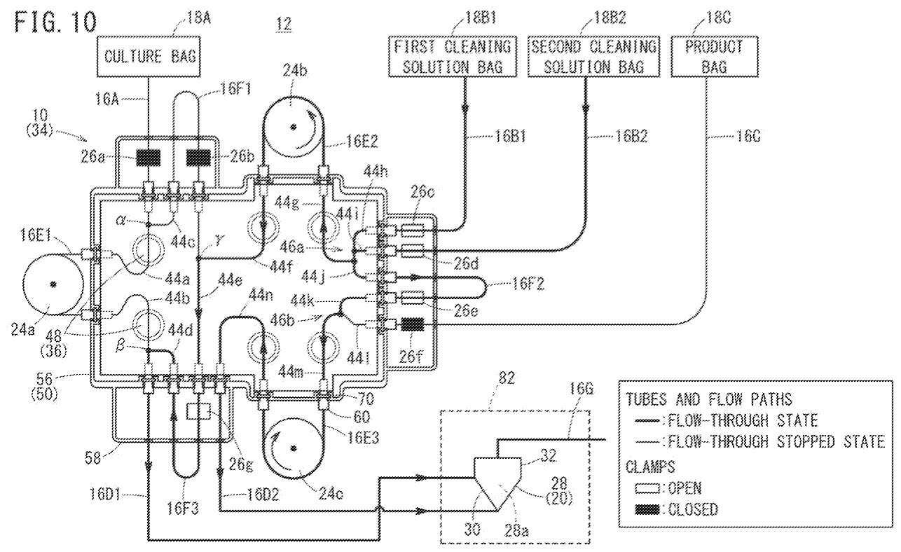

Following the second culture supplying step, the cleaning process may further include, as illustrated in FIG. 10, further supplying the cleaning solution to the treatment case 28 to clean the cells (remove the medium). For example, in the cleaning step, the cell cleaning device 14 may place in communication a path from the first and second cleaning solution bags 18B1, 18B2 to the treatment case 28. The third clamp 26c, the fourth clamp 26d, the fifth clamp 26e, and the seventh clamp 26g may be opened, while the first clamp 26a, the second clamp 26b, and the sixth clamp 26f may be closed. Further, by the cell cleaning device 14 driving the second and third pumps 24b and 24c, the cleaning solution of the first and second cleaning solution bags 18B1 and 18B2 may pass through two paths—the first and second treatment tubes 16D1 and 16D2)—and may be supplied to the treatment case 28. The cell cleaning device 14 may also cause the rotor 82 to rotate and applies a centrifugal force to the treatment case 28.

The cleaning solution in the first and second cleaning solution bags 18B1 and 18B2 may flow into the flow paths 44 (e.g., the eighth path 44h and the ninth path 44i) of the flow path cassette 10 through each of the first and second cleaning solution tubes 16B1 and 16B2 and through the first flow path switching unit 46a. In certain variations, the cleaning solution may also pass through the seventh path 44g, the second pump tube 16E2, the sixth path 44f, the fifth path 44e, the third clamp tube 16F3, the fourth path 44d, and/or the second path 44b, and then may flow into the first treatment tube 16D1, whereupon the cleaning solution may flow into the internal space 28a from the side of the treatment case 28. In certain variations, the cleaning solution may pass through the tenth path 44j, the second clamp tube 16F2, the eleventh path 44k, the thirteenth path 44m, the third pump tube 16E3, and/or the fourteenth path 44n, and may flow into the second treatment tube 16D2, whereupon the cleaning solution may flow into the internal space 28a from the apex of the treatment case 28. As such, inside the treatment case 28, removal of the medium that was subjected to centrifugation further progresses and the cell concentration is increased.

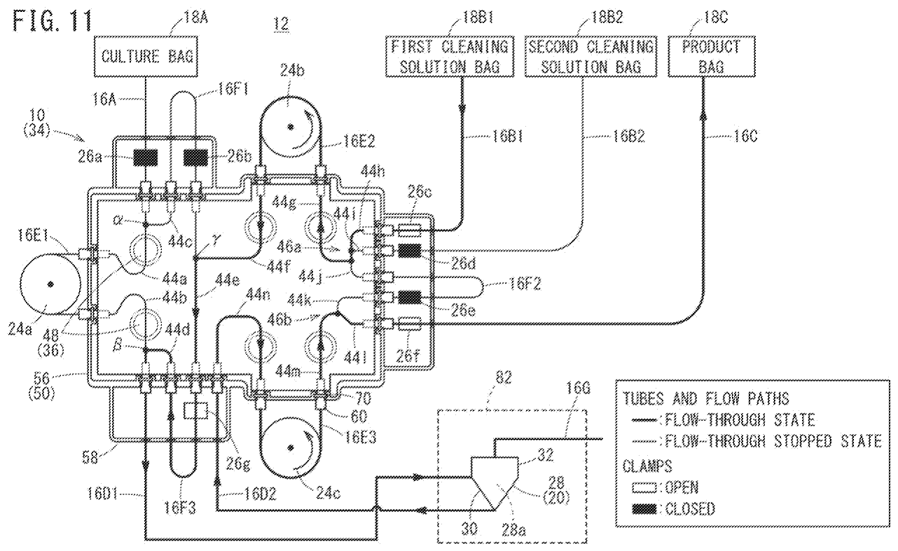

After completion of the cleaning step, the cleaning process may further include treating the cells. For example, as illustrated in FIG. 11, a product transfer step may be executed to transfer the cleaned cells inside the treatment case 28 into the product bag 18C. In the product transfer step, the cell cleaning device 14 may place in communication a path from the treatment case 28 to the product bag 18C, together with placing in communication a path from the first cleaning solution bag 18B1 to the treatment case 28. For this purpose, in the cell cleaning device 14, the third clamp 26c, the sixth clamp 26f, and the seventh clamp 26g may be opened, while the first clamp 26a, the second clamp 26b, the fourth clamp 26d, and the fifth clamp 26e may be closed. In such instances, the cell cleaning device 14 may drive the second and third pumps 24b and 24c, together with causing the rotor 82 to rotate at a lower speed of rotation (e.g., about 2400 rpm).

The cleaning solution in the first cleaning solution bag 18B1 may pass through the first cleaning solution tube 16B1, the eighth path 44h, the seventh path 44g, the second pump tube 16E2, the sixth path 44f, the fifth path 44e, the third clamp tube 16F3, the fourth path 44d, and/or the second path 44b, and may flow into the first treatment tube 16D1, whereupon the cleaning solution may flow into the internal space 28a from the side of the treatment case 28. Further, the cells inside the treatment case 28 (including the cleaning solution) may pass through the second treatment tube 16D2, the fourteenth path 44n, the third pump tube 16E3, the thirteenth path 44m, and the twelfth path 44l, and may flow to the product tube 16C, whereupon the cells may flow into the product bag 18C from the product tube 16C. In the product transfer step, the flow rate of the cleaning solution by the second pump 24b, and the flow rate of the product (cells) by the third pump 24c, may be the same rate (e.g., about 40 mL/min).

By the above process, the cleaned cells (highly concentrated cells) may be stored in the product bag 18C, whereupon the cleaning process of the cell cleaning system 22 may be brought to an end. After completion of the cleaning process, the operator may cut and seal the product tube 16C of the cleaning kit 12, and may separate the product bag 18C from the cleaning kit 12. The cleaning kit 12 including the flow path cassette 10 may be removed from the cell cleaning device 14 and may be discarded.

In the aforementioned cleaning process, the pressure may be detected in each of the pressure detecting units 36 of the respective flow paths 44 (i.e., the first path 44a, the second path 44b, the sixth path 44f, the seventh path 44g, the thirteenth path 44m, and/or the fourteenth path 44n) of the cell cleaning system 22. As illustrated in FIG. 5, in each of the pressure detecting units 36, the deformation preventative member 90 prevents the first bulging portion 49a1 of the first sheet 42a from being displaced and from being crushed due to the negative pressure.

Accordingly, in the case that liquid is not flowing in the flow chambers 48a of the detection channel portions 48, a pressure may not be applied to the second bulging portions 49a2 from the interiors of the flow chambers 48a, and the flat portions 49b may undergo almost no displacement. Therefore, the load detecting units 106 may detect a weak pressure. On the other hand, in the case that liquid is flowing in the flow chambers 48a of the detection channel portions 48, a pressure may act on the second bulging portions 49a2 from the liquid of the flow chambers 48a, whereby the plate members 102 may be displaced in a direction to approach the magnets 104. As such, the load detecting units 106 may detect the loads of the magnets 104 in accordance with the displacement of the plate members 102, and may transmit the same to the control unit 88 of the cell cleaning device 14.

In the case that a positive pressure acts on the flow chambers 48a, since the first sheet 42a may be supported as a result of being in contact with the deformation preventative members 90 and the flow path cassette 10, the first bulging portions 49a1 of the first sheet 42a of the detection channel portions 48 may not be displaced. As a result, the flat portions 49b of the second sheet 42b side may be significantly and reliably displaced, whereby the detection of pressure by the load cells 100 can be suitably performed.

Further, in the case that a negative pressure acts on the flow chambers 48a, since the first sheet 42a may be supported so as not to be crushed by the deformation preventative members 90 and the outer peripheral portions 98 may be in contact with each other, the first bulging portions 49a1 of the first sheet 42a may not displaced. As a result, the flat portions 49b of the second sheet 42b may be displaced by the action of the negative pressure, and detection of the negative pressure by the load cells 100 may be performed.

The present invention is not limited to the above-described embodiment, and various modifications can be adopted in accordance with the essence and gist of the present invention. For example, it should be recognized that the shapes of the respective flow paths 44 of the flow path cassette 10 may be freely designed. Further, the flow path cassette 10 may be applied to a cell culture device including a bioreactor.

Further, the flow path cassette 10 is not limited to one that is used for treatment of cells having been cultured in a cell culture system and may be used in a blood collection bag system for extracting blood cell components, lymphocyte components, or the like.

Technical concepts and effects that can be grasped from the above-described embodiments will be described below.

One aspect of the present invention may be characterized by the flow path cassette 10 that may include the first sheet 42a and the second sheet 42b, which may be superimposed on and joined to each other. The first sheet 42a and the second sheet 42 may include flexible materials. The flow path cassette 10 may include flow paths 44 disposed between the first and second sheets 42a, 42b and detection channel portions 48 may be disposed along the flow paths 44. For example, the detection channel portions 48 may be disposed midway along the respective flow paths 44. The detection channel portions 48 may include the first bulging portions 49a1. The first bulging portions 49a1 may be wider in the planar direction than the flow path 44 and may bulge in the thickness direction from the first sheet 42a. The second bulging portions 49a2 may be formed in the second sheet 42b and may face the first bulging portions 49a1. The second bulging portions may bulge in the thickness direction from the second sheet 42b, and the plate members 102 may be disposed on the second bulging portions 49a2. The deformation preventative members 90, which may be made of a material that is harder than the first sheet 42a, may be disposed along the outer surfaces of the first bulging portions 49a1 of the first sheet 42a, and may be joined to the outer surfaces of the first bulging portions 49a1 of the first sheet 42a. The deformation preventative members 90 may prevent deformation to the first bulging portions 49a1.

The deformation preventative members 90 may prevent the detection channel portions 48 from being displaced or crushed, and using the load cells 100 that may be disposed in proximity to the detection channel portions 48, both positive and negative pressures acting on the flow paths 44 can be measured.

In various aspects, the flow path cassette 10 may include the frame 50 that supports the first sheet 42a and the second sheet 42b. The frame 50 may include the plate-shaped cover portion 54 disposed in facing relation to the first sheet 42a, and the deformation preventative members 90 may be configured to prevent displacement of the first sheet 42a by coming into contact with the cover portion 54 when a positive pressure is applied to the flow paths 44. The flat portions 49b of the second sheet 42b may be significantly and reliably displaced in accordance with the internal pressure of the flow paths 44, whereby the detection of pressure by the load cells 100 can be suitably performed.

The deformation preventative members 90 may be formed in a dome shape along outer surfaces of the first bulging portions 49a1. As such, the thickness of the deformation preventative members 90 can be reduced, and the deformation preventative members 90 may be made lighter in weight.

The plate members 102 may cause loads to be applied to the load cells 100 in accordance with the internal pressure of the flow path 44, and the plate members 102 may be made of a magnetic material that may be capable of being attracted to the magnets 104 that may be disposed on the load cells 100. AS such, a load change can be made to occur in the load cells 100 not only by a positive pressure, but also by a negative pressure.

The outer peripheral portions 98 of the deformation preventative members 90 may be in contact with the joined portions of the first sheet 42a and the second sheet 42b that may be outside the first bulging portions 49a1. As such, even in the case that a negative pressure has occurred in the flow paths 44, the first sheet 42a can be prevented from being deformed due to crushing or collapsing of the first bulging portions 49a1, and the negative pressure can be reliably measured.

Another aspect of the present invention may be characterized by the cleaning kit 12 which includes the flow path cassette 10 integrated with the flow paths 44, the plurality of bags 18 connected to the flow path cassette 10, and the tubes 16 connected to the flow path cassette 10. In the cleaning kit 12, the flow path cassette 10 may further include the first sheet 42a and the second sheet 42b, which can be made from a flexible material, and may be superimposed on each other and joined. The flow paths 44 may be disposed between the first sheet 42a and the second sheet 42b, and the detection channel portions 48 may be disposed midway along the flow paths 44. The detection channel portions 48 may include the first and second bulging portions 49a1 and 49a2 that may be wider in the planar direction than the flow paths 44 and which bulge in the thickness direction from the first sheet 42a and the second sheet 42b, the plate members 102 disposed on the second bulging portions 49a2 of the second sheet 42b, and the deformation preventative members 90 which may be made of a material that may be harder than the first sheet 42a, form shapes along the outer surfaces of the first bulging portions 49a1 of the first sheet 42a, and may be joined to the outer surfaces of the first bulging portions 49a1 of the first sheet 42a.

In accordance with the above-described cleaning kit 12, it is possible to measure the internal pressure of the flow paths 44 of the flow path cassette 10.

Still another aspect of the present invention may be characterized by a cell cleaning system 22 having the cleaning kit 12 including the flow path cassette 10 in which the flow paths 44 may be integrated, the plurality of bags 18 connected to the flow path cassette 10, and the tubes 16 connected to the flow path cassette 10, and a cell cleaning device 14 in which the cleaning kit 12 may be set. In the cell cleaning system 22, the flow path cassette 10 may further include the first sheet 42a and the second sheet 42b, which may be made from a flexible material, and may be superimposed on each other and joined, the flow paths 44 that may be formed between the first sheet 42a and the second sheet 42b, and the detection channel portions 48 which may be disposed midway along the flow paths 44. The detection channel portions 48 may include the first bulging portions 49a1 that may be wider in the planar direction than the flow path 44 and which bulge in the thickness direction from the first sheet 42a, the second bulging portions 49a2 that may formed on portions of the second sheet 42b facing the first bulging portions 49a1 and which bulge in the thickness direction from the second sheet 42b, the plate members 102 disposed on the second bulging portions 49a2 of the second sheet 42b, and the deformation preventative members 90 which may be made of a material that may be harder than the first sheet 42a, form shapes along the outer surfaces of the first bulging portions 49a1 of the first sheet 42a, and may be joined to the outer surfaces of the first bulging portions 49a1 of the first sheet 42a and prevent deformation to the first bulging portions 49a1.

In accordance with the above-described cell cleaning system 22, it is possible to measure the internal pressure of the flow paths 44 of the flow path cassette 10.

1. A flow path cassette comprising:
a first flexible sheet;
a second flexible sheet parallel with the first flexible sheet;
a plurality of flow paths disposed between and surrounded by the first and second flexible sheets; and
a plurality of detection channel portions disposed along one or more flow paths of the plurality of flow paths, wherein each of the detection channel portions comprises:
a first bulging portion;
a second bulging portion opposing the first bulging portion;
a plate member aligned with a portion of the second bulging portion away from the first bulging portion; and
a deformation preventative member aligned with the first bulging portion and configured to prevent deformation of the first bulging portion.

2. The flow path cassette of claim 1, wherein the first bulging portion has a dimension in the planar direction that is larger than a corresponding dimension of the respective flow path.

3. The flow path cassette of claim 1, wherein the first bulging portion bulges in a first direction relative to a major axis of the flow path cassette, and the second bulging portion bulges in a second direction relative to the major axis of the flow path cassette and away from the first direction.

4. The flow path cassette of claim 3, wherein the second bulging portion comprises a center portion and two or more protrusions disposed around the center portion that bulge away from the center portion in the second direction.

5. The flow path cassette of claim 4, wherein the plate member is aligned with the center portion of the second bulging portion.

6. The flow path cassette of claim 5, wherein the plate member is coupled to second bulging portion.

7. The flow path cassette of claim 5, wherein the plate member has a shape corresponding to a shape of the center portion.

8. The flow path cassette of claim 5, wherein a cover sheet extends between the two or more protrusions to enclose the plate member.

9. The flow path cassette of claim 1, wherein the deformation preventative member comprises a material having a first hardness that is greater than a second hardness of the first flexible sheet.

10. The flow path cassette of claim 1, wherein the deformation preventative member is coupled to the first bulging portion.

11. The flow path cassette of claim 1, wherein the first bulging portion is formed in the first flexible sheet, the second bulging portion is formed in the second flexible sheet, and a flow chamber configured to receive the respective flow path is formed between the first bulging portion and the second bulging portion.

12. The flow path cassette of claim 11, wherein the deformation preventative member extends beyond the first bulging portion and contacts two or more areas of the first flexible sheet.

13. The flow path cassette of claim 11, wherein the plate member comprises a magnetophilic metal.

14. The flow path cassette of claim 13, wherein each of the detection channel portions further comprises a load cell comprising one or more magnets disposed adjacent to the plate member, wherein the plate member is displaced is response to an internal pressure of the flow chamber and the displacement causes attractive forces between the one or more magnets and the plate member to change.

15. The flow path cassette of claim 1, further comprising:
a frame configured to support the first flexible sheet and the second flexible sheet, wherein the frame comprises a plate-shaped cover portion aligned with the first flexible sheet, and the deformation preventative member is configured to prevent displacement of the first bulging portion by coming into contact with the cover portion when the flow path has a positive pressure.

16. A flow path cassette comprising:
a first flexible sheet comprising a first bulging portion that bulges in a first direction relative to a major axis of the flow path cassette;
a second flexible sheet parallel with the first flexible sheet and comprising a second bulging portion that faces the first bulging portion and bulges in a second direction relative to the major axis of the flow path cassette, wherein the second bulging portion comprises a center portion and two or more protrusions disposed around the center portion that bulge away from the center portion in the second direction;
a plurality of flow paths disposed between and surrounded by the first and second flexible sheets;
a deformation preventative member aligned with the first bulging portion and configured to prevent deformation of the first bulging portion; and
a ferromagnetic plate member aligned with the center portion and configured to move with the second bulging portion.

17. The flow path cassette of claim 16, wherein the first bulging portion has a dimension in a planar direction that is larger than a corresponding dimension of a respective flow path of the plurality of flow paths.

18. The flow path cassette of claim 16, wherein the deformation preventative member extends beyond the first bulging portion and contacts two or more areas of the first flexible sheet.

19. The flow path cassette of claim 16, further comprising:
a frame configured to support the first flexible sheet and the second flexible sheet, wherein the frame comprises a plate-shaped cover portion aligned with the first flexible sheet, and the deformation preventative member is configured to prevent displacement of the first bulging portion by coming into contact with the cover portion when the flow path has a positive pressure.

20. A flow path cassette comprising:
first flexible sheet comprising a first bulging portion that bulges in a first direction relative to a major axis of the flow path cassette;
a second flexible sheet parallel with the first flexible sheet and comprising a second bulging portion that faces the first bulging portion and bulges in a second direction relative to the major axis of the flow path cassette, wherein the second bulging portion comprises a center portion and two or more protrusions disposed around the center portion that bulge away from the center portion in the second direction;
a plurality of flow paths disposed between and surrounded by the first and second flexible sheets, wherein the first bulging portion has a dimension in a planar direction that is larger than a corresponding dimension of a respective flow path of the plurality of flow paths;
a deformation preventative member aligned with the first bulging portion and configured to prevent deformation of the first bulging portion, wherein the deformation preventative member extends beyond the first bulging portion and contacts two or more areas of the first flexible sheet;
a ferromagnetic plate member aligned with the center portion and configured to move with the second bulging portion; and
a frame configured to support the first flexible sheet and the second flexible sheet, wherein the frame comprises a plate-shaped cover portion aligned with the first flexible sheet, and the deformation preventative member is configured to prevent displacement of the first bulging portion by coming into contact with the cover portion when the flow path has a positive pressure.
